(19) United States
(12) Patent Application Publication
(10) Pub. No.: US 2012/0219633 A1
(43) Pub. Date: Aug. 30, 2012

(54) REMOVAL OF IMMUNOGLOBULINS AND LEUKOCYTES FROM BIOLOGICAL FLUIDS

(75) Inventor: Samuel O. Sowemimo-Coker, Dix Hills, NY (US)

(73) Assignee: PALL CORPORATION, Port Washington, NY (US)

(21) Appl. No.: 13/036,169

(22) Filed: Feb. 28, 2011

(51) Int. Cl. A61K 35/20; B01D 15/00

(52) U.S. Cl. 424/535; 210/663; 210/263

(57) ABSTRACT

Devices, systems, and methods for depleting fluids of immunoglobulins and leukocytes are disclosed.

### BACKGROUND OF THE INVENTION

The presence of undesirable materials such as leukocytes, immunoglobulins, and cytokines in blood and blood components can lead to adverse effects in a patient receiving a transfusion. For example, leukocyte-contaminated transfusions are associated with febrile reactions, alloimmunization, graft versus host disease, and rejection of the transfused red blood cells or platelets. The transfusion of immunoglobulins has been associated with, for example, transfusion-related acute lung injury (TRALI), which is the most common cause of transfusion-related death in the U.S.

While leukocytes can be removed using leukocyte depletion filters, conventional techniques for removing immunoglobulins involve a labor- and reagent-intensive effort, such as controlling the pH to very high or very low levels, specified salt conditions, and careful control of flow rates. Such techniques are not compatible with processing blood and blood components for transfusion into patients.

The present invention provides for ameliorating at least some of the disadvantages of the prior art. These and other advantages of the present invention will be apparent from the description as set forth below.

### BRIEF SUMMARY OF THE INVENTION

An embodiment of the invention provides a device for removing immunoglobulins and leukocytes from a biological fluid, the device comprising a biological fluid container containing immunoglobulin binding media and a porous fibrous leukocyte depletion medium therein.

In another embodiment, a biological fluid filter device is provided, the device comprising a housing including an inlet and an outlet and defining a fluid flow path between the inlet and the outlet, and a porous fibrous leukocyte depletion filter disposed in the housing across the fluid flow path, the device further comprising a chamber for receiving immunoglobulin binding media.

In another embodiment, a system for removing immunoglobulins and leukocytes from a biological fluid is provided, the system comprising (a) a biological fluid container, containing immunoglobulin binding media therein; and, (b) a leukocyte depletion device comprising a housing having an inlet and an outlet and defining a fluid flow path between the inlet and the outlet and having a porous fibrous leukocyte depletion filter disposed between the inlet and the outlet and across the fluid flow path; wherein the leukocyte depletion device is downstream of, and in fluid communication with, the biological fluid container.

In accordance with an embodiment of the present invention, a method for processing biological fluid comprises depleting immunoglobulins and leukocytes from the fluid, in some embodiments, the method further comprises providing the immunoglobulin- and leukocyte-depleted biological fluid to a subject, preferably, a mammal.

A method for processing biological fluid according to an embodiment of the invention comprises placing the biological fluid in contact with immunoglobulin-specific binding media; and, passing the biological fluid through a porous fibrous leukocyte depletion filter to obtain immunoglobulin- and leukocyte-depleted biological fluid.

In yet another embodiment of a method for processing biological fluid the method comprises placing the biological fluid in contact with immunoglobulin-specific binding media to obtain immunoglobulin-depleted biological fluid; and, passing the immunoglobulin-depleted biological fluid through a porous fibrous leukocyte depletion filter to obtain immunoglobulin- and leukocyte-depleted biological fluid.

In another embodiment, a biological fluid product is provided, wherein the product has been depleted of immunoglobulins and leukocytes according to the invention. Preferably, the product is suitable for use as a transfusion product, e.g., for humans and animals such as horses.

### BRIEF DESCRIPTION OF THE SEVERAL VIEWS OF THE DRAWING(S)

FIG. 1 is a diagrammatic illustration of an embodiment of a system according to the present invention, comprising a first container for receiving biological fluid, the first container containing immunoglobulin binding media, and downstream of the first container, a filter device comprising an immunoglobulin binding media chamber (for receiving immunoglobulin binding media passed from the first container) and a leukocyte depletion filter comprising a leukocyte depletion element, the system further comprising a second container for receiving immunoglobulin- and leukocyte-depleted biological fluid, wherein the second container is downstream of the filter device.

FIG. 2 shows views of one embodiment of a filter device according to the present invention (for use in the embodiment of the system shown in FIG. 1), wherein the filter device comprises an inlet portion, an outlet portion, and an internal ring, and the device includes a chamber for receiving immunoglobulin binding media, as well as a leukocyte depletion filter comprising a leukocyte depletion filter element. FIG. 2A shows a cross-sectional side view of the assembled device; FIG. 2B shows various views of the inlet portion and the outlet portion.

FIG. 3 is a diagrammatic illustration of an embodiment of a system according to the present invention, comprising a first container for receiving biological fluid, the first container comprising a mesh bag containing immunoglobulin binding media therein, and downstream of the first container, a filter device comprising a leukocyte depletion filter, the system further comprising a second container for receiving immunoglobulin- and leukocyte-depleted biological fluid, wherein the second container is downstream of the filter device.

FIG. 4 is a diagrammatic illustration of another embodiment of a system according to the present invention, comprising a first container for receiving biological fluid, the first container comprising a filter comprising immunoglobulin binding media and a leukocyte depletion filter element arranged to provide a chamber for the immunoglobulin binding media, the system further comprising a second container for receiving immunoglobulin- and leukocyte-depleted biological fluid.

### DETAILED DESCRIPTION OF THE INVENTION

Advantageously, in accordance with the invention, immunoglobulins and leukocytes can be removed from biological fluids under physiologic conditions of pH and salt concentration, which are compatible with normal physiologic functions of blood and blood components such as plasma, red blood cells, and platelet concentrates. Other advantages are that the invention can be carried out without a labor intensive effort of packing adsorbent materials into special chromatographic columns and no special equipment is required for controlling flow rates or binding kinetics. Moreover, biological fluids can be depleted of immunoglobulins and leukocytes in a relatively short period of time, e.g., about 1 hour or less, preferably, about 45 minutes or less.

A variety of immunoglobulins can be bound to the immunoglobulin binding media in accordance with the invention, e.g., whole immunoglobulins, including monoclonal and polyclonal antibodies, as well as the heavy chains and/or light chains and/or the fragments thereof, e.g., Fab, F(ab′)₂, F_{c} and Fᵥ. In particular, IgG can be bound. Alternatively, or additionally, one or more of any of the following: IgA, IgM, IgD and IgE, can be bound. Alternatively or additionally, in some embodiments, other undesirable materials, e.g., cytokines and/or pathogens (e.g., prions) can be bound to the media and removed.

In accordance with an embodiment of the present invention, a method for processing biological fluid comprises depleting immunoglobulins and leukocytes from the fluid and obtaining an immunoglobulin- and leukocyte-depleted biological fluid; in some embodiments, the method further comprises administering the immunoglobulin- and leukocyte-depleted biological fluid to a subject, preferably, a mammal.

In accordance with another embodiment of the present invention, a method for removing immunoglobulins and leukocytes from a biological fluid is provided, the method comprising (a) placing the biological fluid in contact with immunoglobulin-specific binding media; and, (b) passing the biological fluid through a porous fibrous leukocyte depletion filter to obtain immunoglobulin- and leukocyte-depleted biological fluid.

In another embodiment, a method for removing immunoglobulins and leukocytes from a biological fluid comprises (a) placing the biological fluid in contact with immunoglobulin-specific binding media to obtain immunoglobulin-depleted biological fluid; and, (b) passing the immunoglobulin-depleted biological fluid through a porous fibrous leukocyte depletion filter to obtain immunoglobulin- and leukocyte-depleted biological fluid.

In another embodiment, a method for reducing or preventing red cell hemolysis in horses comprises obtaining biological fluid or colostrum from a mare, depleting immunoglobulins and leukocytes from the biological fluid or the colostrum to obtain an immunoglobulin- and leukocyte-depleted biological fluid or an immunoglobulin- and leukocyte-depleted colostrum, and administering the immunoglobulin- and leukocyte-depleted biological fluid or immunoglobulin- and leukocyte-depleted colostrum to a foal.

Preferably, the immunoglobulin-specific binding media comprise beads, and an embodiment of the method comprises placing the biological fluid in contact with the beads in a flexible container, such as a flexible blood bag.

In an embodiment, the method comprises passing the biological fluid from the flexible blood bag and through a filter device comprising a housing having an inlet and an outlet and defining a fluid flow path between the inlet and the outlet wherein the porous fibrous leukocyte depletion filter is disposed in the housing and across the fluid flow path.

An embodiment of the method can include retaining beads in a bead-receiving chamber of the filter device as the biological fluid passes through the device. Alternatively, an embodiment of the method can include retaining beads in a flexible blood bag including a porous element that retains the beads as the biological fluid passes from the bag and toward the filter device.

In some embodiments of the method, placing the biological fluid in contact with immunoglobulin-specific binding media also includes placing the biological fluid in contact with cytokine-specific binding media, and deleting immunoglobulins and at least one cytokine from the biological fluid.

In another embodiment, a device for removing immunoglobulins and leukocytes from a biological fluid comprises a biological fluid container containing immunoglobulin-specific binding media and a porous fibrous leukocyte depletion filter therein. In an embodiment of the device, the biological fluid container comprises a flexible container having walls comprising a flexible film, and the porous fibrous leukocyte depletion medium is arranged to provide a hollow structure having at least one closed end, the hollow structure containing the immunoglobulin-specific binding media therein.

A filter device according to another embodiment of the invention comprises a housing including an inlet and an outlet and defining a fluid flow path between the inlet and the outlet, an internal ring, and a leukocyte depletion filter comprising a leukocyte depletion filter element disposed in the housing across the fluid flow path, the leukocyte depletion filter having an upstream surface facing the inlet, and a downstream surface facing the outlet, the device further comprising an upstream chamber for receiving immunoglobulin binding media, the chamber having a side wall defined by the internal ring, wherein the chamber is arranged to receive immunoglobulin binding media passing through the inlet. In an embodiment, the device further comprises a porous element such as a mesh or screen element, upstream of the upstream surface of the leukocyte depletion filter element, e.g., wherein the mesh or screen element provides a bottom wall of the chamber for receiving immunoglobulin binding media. For example, the mesh or screen element can be interposed between the internal ring and the upstream surface of the leukocyte depletion filter element.

Alternatively, or additionally, in some embodiments of the device, the internal ring is arranged such that the received immunoglobulin binding media form a packed column-like matrix within the housing.

A system for removing immunoglobulins and leukocytes from a biological fluid according to an embodiment of the system comprises (a) a biological fluid container, containing immunoglobulin-specific binding media therein; and (b) a leukocyte depletion device comprising a housing having an inlet and an outlet and defining a fluid flow path between the inlet and the outlet and having a porous fibrous leukocyte depletion filter disposed between the inlet and the outlet and across the fluid flow path; wherein the leukocyte depletion device is downstream of, and in fluid communication with, the biological fluid container.

In another embodiment, a biological fluid product is provided, wherein the biological fluid has been depleted of immunoglobulins and leukocytes according to the invention. Preferably, the product is suitable for use as a transfusion product, e.g., for humans and animals such as horses.

In yet another embodiment, a colostrum product is provided, wherein the colostrum has been depleted of immunoglobulins and leukocytes according to the invention. Preferably, the product is suitable for administration to animals such as horses.

Embodiments of the device and system are suitable for a variety of applications, including administration of biological fluids to humans and to animals, and testing compatibilities of biological fluids. The biological fluid can be from a number of sources, preferably mammals. It is preferred that the mammals are from the order Carnivora, including Felines (cats) and Canines (dogs), more preferably from the order Artiodactyla, including Bovines (cows) and Swines (pigs) or of the order Perssodactyla, including Equines (horses). For example, embodiments of the invention can be used to process biological fluids to be administered to horses, e.g., to prevent or reduce red cell lysis, for example, to prevent or reduce Neonatal Isoerythrolysis in foals.

Typically, the mammals are of the order Primates, Ceboids, or Simoids (monkeys) or of the order Anthropoids (humans and apes). An especially preferred mammal is the human.

Each of the components of the invention will now be described in more detail below, wherein like components have like reference numbers.

The following definitions are used in accordance with the invention.

Biological Fluid. A biological fluid includes any treated or untreated fluid associated with living organisms, particularly blood, including whole blood, warm or cold blood, cord blood, and stored or fresh blood; treated blood, such as blood diluted with at least one physiological solution, including but not limited to saline, nutrient, and/or anticoagulant solutions; blood components, such as platelet concentrate (PC), platelet-rich plasma (PRP), platelet-poor plasma (PPP), platelet-free plasma, plasma, fresh frozen plasma (FFP), components obtained from plasma, packed red cells (PRC), transition zone material or buffy coat (BC); blood products derived from blood or a blood component or derived from bone marrow; stem cells; red cells separated from plasma and resuspended in physiological fluid or a cryoprotective fluid; and platelets separated from plasma and resuspended in physiological fluid or a cryoprotective fluid. A biological fluid also includes a physiological solution comprising a bone marrow aspirate. The biological fluid may have been treated to remove some of the leukocytes before being processed according to the invention. As used herein, blood product or biological fluid refers to the components described above, and to similar blood products or biological fluids obtained by other means and with similar properties.

A “unit” is the quantity of biological fluid from a donor or derived from one unit of whole blood. It may also refer to the quantity drawn during a single donation. Typically, the volume of a unit varies, the amount differing from patient to patient and from donation to donation. Multiple units of some blood components, particularly platelets and buffy coat, may be pooled or combined, typically by combining four or more units.

As used herein, the term “closed” refers to a system that allows the collection and processing (and, if desired, the manipulation, e.g., separation of portions, separation into components, filtration, storage, and preservation) of biological fluid, e.g., donor blood, blood samples, and/or blood components, without the need to compromise the sterile integrity of the system. A closed system can be as originally made, or result from the connection of system components using what are known as “sterile docking” devices. Illustrative sterile docking devices are disclosed in, for example, U.S. Pat. Nos. 4,507,119, 4,737,214, and 4,913,756.

A variety of immunoglobulin-specific binding media are known in the art, and can be used in accordance with the invention. The binding media can be any suitable material, with the limitation that the material does not substantially adversely affect the desired biological fluid components or the desired colostrum components present in the product, e.g., a transfusion product. For example, with respect to biological fluid, the binding media does not substantially adversely affect one or more of the following: red blood cells, platelets, plasma, and plasma proteins. With respect to the colostrum, while the binding media removes immunoglobulins, the binding media does not substantially affect the non-immunoglobulin colostrum proteins. Preferably, the media comprise adsorbent particles that are roughly spherical, such as beads, e.g., organic materials such cellulose, starch, agar, dextran or agarose (e.g., including Sepharose™); hydrophilic synthetic polymers, including substituted or unsubstituted polyacrylamides, polymethacrylamides, polyacrylates, polymethacrylates, polyvinyl hydrophilic polymers such as polyvinyl alcohol, polystyrene, polysulfone, and copolymers or styrene and divinylbenzene, and mixtures thereof. Alternatively, inorganic materials may be used, including, but are not limited, to mineral materials, such as silica; hydrogel-containing silica, zirconia, titania, alumina; and other ceramic materials. It is also possible to use mixtures of these materials, or composite materials formed by copolymerization, or other types of beads, although fibrous media and membranes can also be used. The adsorbent particles can be, for example, made of hydrophilic resins, hydrophobic resins, ion exchange resins, or activated carbon.

In those embodiments wherein the binding media are adsorbent particles, the particles, e.g., beads, are preferably porous. The beads, particularly porous beads, may have a high surface area, for example, at least about 40 m²/g to about 700 m²/g, although in some embodiments, the surface area can be less than about 40 m²/g or more than about 700 m²/g. Typically, the porous beads have a surface area of at least about 50 m²/g. The particles can be any suitable diameter. Typically, the particles are about 500 micrometers (μm) in diameter or less, more typically, about 150 micrometers in diameter or less, e.g., in the range from about 10 micrometers to about 500 micrometers in diameter.

Typically, in those embodiments wherein the binding media are adsorbent particles, and a unit of biological fluid is to be treated, about 2 to about 500 g of particles are utilized.

The binding media are typically treated or modified, e.g., with a variety of functional groups (e.g., ionic, hydrophobic, acidic, basic) and/or linked to a ligand, to, for example, provide the desired binding specificity. In one illustrative embodiment, the adsorbent particulate media comprises 4-Mercapto-Ethyl-Pyridine (4-MEP) HyperCel™ chromatography sorbent (Pall Corporation, NY); in other illustrative embodiments, the adsorbent particulate media comprises phenylpropylamine (PPA) HyperCel™ chromatography sorbent (Pall Corporation, NY); or chromatography sorbents with Protein A and/or Protein G linked thereto, hydroxyapatite HA Ultrogel®, triazine based protein A mimetics (Prometic), and/or the media are prepared as described in International Publication No. WO 2005/073711, International Publication No. WO 2004/024318; U.S. Pat. No. 6,498,236, or U.S. Pat. No. 7,144,743. In some embodiments, a combination of chemistries is used.

Typically, while the particles can be retained in a container (e.g., retained within a mesh or screen bag or pouch, or the container can comprise a porous element such as a mesh or screen preventing passage of the particles through a port of the container), or within the cavity of a filter element, or in a chamber of a filter device, individual particles are loose, not immobilized by binding to a matrix. However, in some embodiments, the particles are immobilized by binding to a matrix.

A variety of porous leukocyte depletion filters, porous leukocyte depletion filter elements, and porous leukocyte depletion media are suitable for use in the invention. In one illustrated embodiment, the porous fibrous leukocyte depletion filter comprises at least one porous fibrous leukocyte depletion element comprising at least one porous fibrous leukocyte depletion medium, wherein the medium can comprise one or more layers of media. The filter can include a plurality of filter elements. The filter can include additional elements, layers, or components, that can have different structures and/or functions, e.g., at least one of prefiltration, support, drainage, spacing and cushioning. Illustratively, the filter can also include at least one additional element such as a mesh and/or a screen.

A variety of materials can be used, including synthetic polymeric materials, to produce the fibrous porous media of the filter elements according to the invention. Suitable synthetic polymeric materials include, for example, polybutylene terephthalate (PBT), polyethylene, polyethylene terephthalate (PET), polypropylene, polymethylpentene, polyvinylidene fluoride, polysulfone, polyethersulfone, nylon 6, nylon 66, nylon 6T, nylon 612, nylon 11, and nylon 6 copolymers, wherein polyesters, e.g., PBT and PET, are more preferred. Typically, the fibrous porous media are prepared from melt-blown fibers. For example, U.S. Pat. Nos. 4,880,548; 4,925,572, 5,152,905, and 6,074,869, disclose porous filter elements prepared from melt-blown fibers.

The filter element can have any desired critical wetting surface tension (CWST, as defined in, for example, U.S. Pat. No. 4,925,572). The CWST can be selected as is known in the art, e.g., as additionally disclosed in, for example, U.S. Pat. Nos. 5,152,905, 5,443,743, 5,472,621, and 6,074,869. Typically, the filter element has a CWST of greater than about 53 dynes/cm (about 53×10⁻⁵ N/cm), more typically greater than about 58 dynes/cm (about 58×10⁻⁵ N/cm), and can have a CWST of about 66 dynes/cm (about 66×10⁻⁵N/cm) or more. In some embodiments, the element may have a CWST in the range from about 62 dynes/cm to about 115 dynes/cm (about 62 to about 162×10⁻⁵ N/cm).

In some embodiments, at least one filter element has a negative zeta potential at physiological pH (e.g., about 7 to about 7.4). For example, a filter element can have a zeta potential of about −3 millivolts (mv), at physiological pH, or the zeta potential can be more negative, e.g., in the range of from about −5 my to about −25 my.

The surface characteristics of the filter element can be modified (e.g., to affect the CWST, to include a surface charge, e.g., a positive or negative charge, and/or to alter the polarity or hydrophilicity of the surface) by wet or dry oxidation, by coating or depositing a polymer on the surface, or by a grafting reaction. Modifications include, e.g., irradiation, a polar or charged monomer, coating and/or curing the surface with a charged polymer, and carrying out chemical modification to attach functional groups on the surface. Grafting reactions may be activated by exposure to an energy source such as gas plasma, vapor plasma, corona discharge, heat, a Van der Graff generator, ultraviolet light, electron beam, or to various other forms of radiation, or by surface etching or deposition using a plasma treatment.

A filter element can have any suitable pore structure, e.g., a pore size (for example, as evidenced by bubble point, or by K_{L} as described in, for example, U.S. Pat. No. 4,340,479, or evidenced by capillary condensation flow porometry), a pore rating, a pore diameter (e.g., when characterized using the modified OSU F2 test as described in, for example, U.S. Pat. No. 4,925,572), or removal rating that reduces or allows the passage therethrough of one or more materials of interest as the fluid is passed through the element. While it is believed leukocytes are primarily removed by adsorption, they can also be removed by filtration. The pore structure can be selected to remove at least some level of leukocytes, while allowing the passing therethrough of desired components, e.g., at least one of plasma, platelets, and red blood cells. The pore structure used depends on the composition of the fluid to be treated, and the desired effluent level of the treated fluid.

A filter element can have a variety of configurations, e.g., substantially planar, corrugated, cylindrical, hollow cylindrical, pouch-like, bag-like, sock-like, or a combination of configurations. In one embodiment, as noted in more detail below, at least one filter element can be arranged in the general form of a pouch, bag, sock or tube having a closed end and an open end (e.g., wherein a conduit passes through the open end and the filter element is sealed such that fluid enters the filter through the conduit).

The filter, in some embodiments comprising a plurality of filter elements is typically disposed in a housing comprising at least one inlet and at least one outlet and defining at least one fluid flow path between the inlet and the outlet, wherein the filter is across the fluid flow path, to provide a filter device. In some embodiments, the filter device includes an immunoglobulin binding media receiving chamber. Alternatively, or additionally, immunoglobulin binding media can be disposed in the housing. Preferably, the filter device is sterilizable. Any housing of suitable shape and providing at least one inlet and at least one outlet may be employed.

In some embodiments, the housing can be fabricated from any suitable rigid impervious material, including any impervious thermoplastic material, which is compatible with the biological fluid being processed. For example, the housing can be fabricated from a polymer. In a preferred embodiment, the housing is a polymer, more preferably a transparent or translucent polymer, such as an acrylic, polypropylene, polystyrene, or a polycarbonated resin. Such a housing is easily and economically fabricated, and allows observation of the passage of the biological fluid through the housing. Suitable housings include, but are not limited to, those disclosed in U.S. Pat. Nos. 4,880,548, 4,25,572, 5,660,731 and 6,231,770.

In some embodiments, as noted in more detail below, the housings further comprise one or more immunoglobulin binding media retaining structures, such as at least one internal ring and/or a support such as a screen or mesh. Typically, the internal ring is fabricated from the same material as the housing, e.g., an acrylic, polypropylene, polystyrene, or a polycarbonated resin.

In one embodiment, the filter device comprises a housing including an inlet and an outlet and defining a fluid flow path between the inlet and the outlet, an internal ring, and a biological fluid filter comprising one or more filter elements, preferably wherein at least one filter element comprises a leukocyte depletion filter element, disposed in the housing across the fluid flow path, the filter having an upstream surface facing the inlet, and a downstream surface facing the outlet, the device further comprising an upstream (of the filter) chamber for receiving immunoglobulin binding media, the chamber having a side wall defined by the internal ring, wherein the chamber is arranged to receive immunoglobulin binding media passing through the inlet.

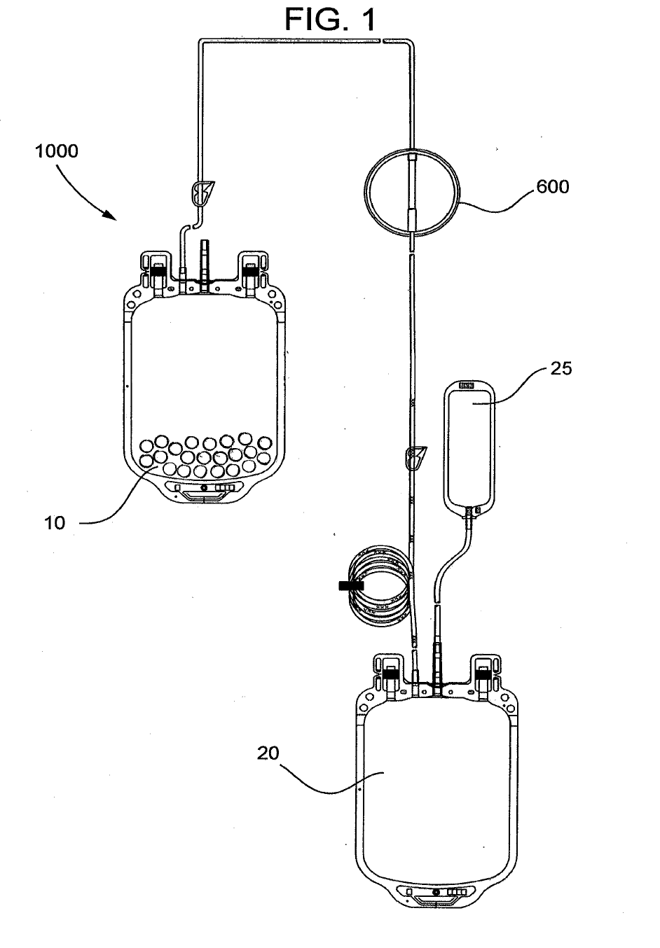

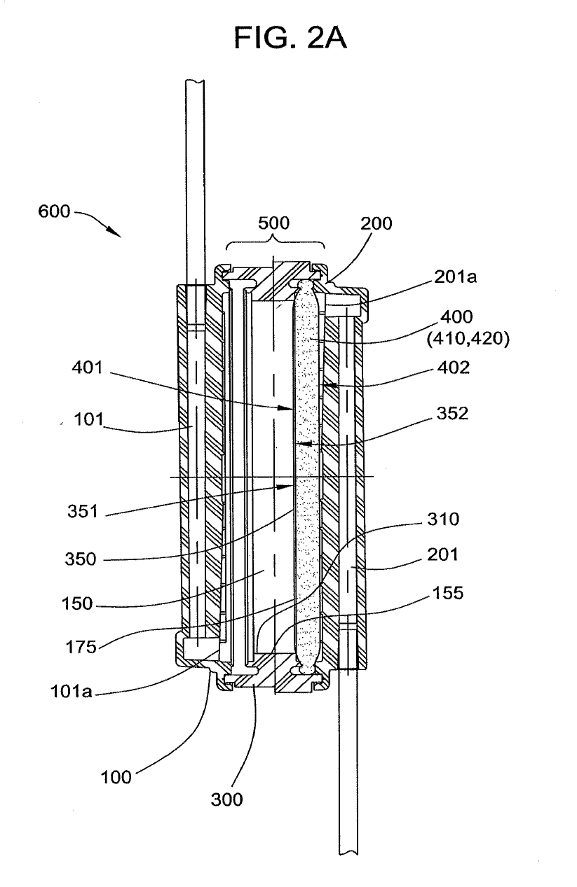

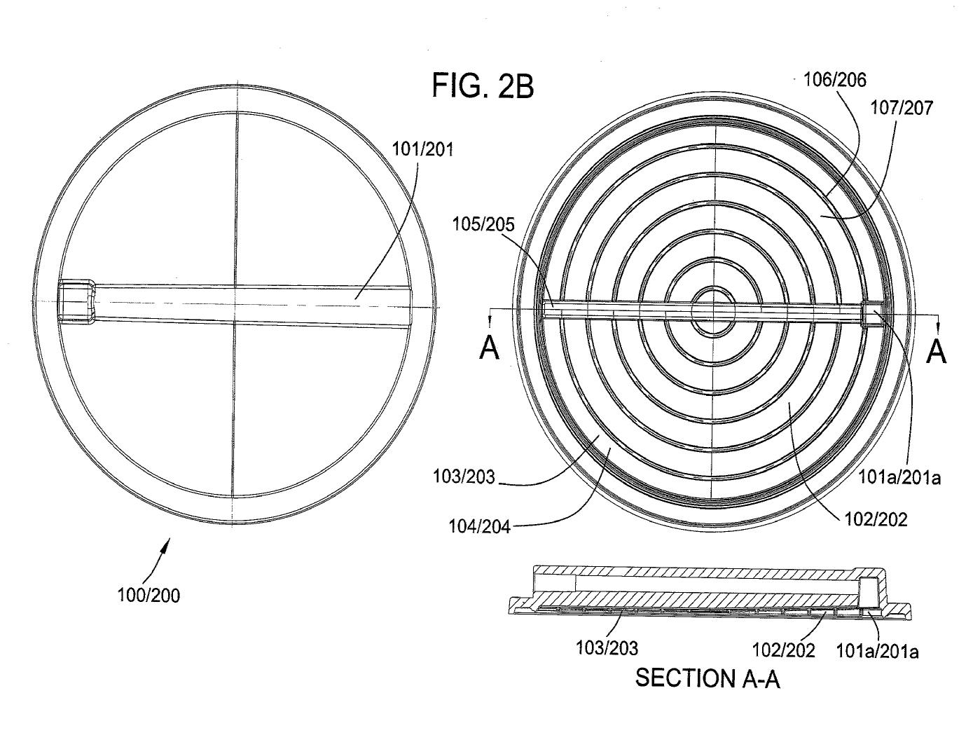

For example, FIG. 2 illustrates an embodiment of a filter device 600 (for use in the system 1000 shown in FIG. 1), the filter device 600 comprising a housing 500 comprising an inlet portion 100 having an inlet 101, an inlet port 101a, an optional inlet channel 107 communicating with the inlet port 101a, an outlet portion 200 having an outlet 201, an outlet port 201a, an optional outlet channel 207 communicating with the outlet port, and an internal ring 300 having an internal side wall 310, and defining a fluid flow path between the inlet and the outlet. The illustrated filter device further comprises a leukocyte depletion filter 400 comprising a porous leukocyte depletion element 410 comprising a porous fibrous leukocyte depletion medium 420, wherein the leukocyte depletion filter 400 comprises an upstream surface 401 and a downstream surface 402, and the leukocyte depletion filter 400 is disposed in the housing across the fluid flow path. In this illustrated embodiment, the device also includes an optional porous element 350 (such as a mesh or screen) upstream of the upstream surface 401 of the leukocyte depletion filter element 400, the mesh having an upstream surface 351 and a downstream surface 352, wherein the device further comprises a chamber 150 for receiving immunoglobulin-specific binding media passing through the inlet and inlet port, the chamber having a chamber side wall 155 defined by the internal side wall 310 of the internal ring 300, and a chamber bottom wall 175 defined by the upstream surface 351 of the mesh 350. In accordance with the invention, the filter can include a plurality of filter elements, for example, the filter can include a prefilter element (not shown) upstream of the leukocyte depletion filter element. In those embodiments wherein the device includes a filter comprising a prefilter element upstream of the leukocyte depletion filter element, and the device further a mesh, the mesh is arranged upstream of the prefilter element.

In the embodiment illustrated in FIG. 2, the ring 300 is interposed between the inlet portion 100 of the housing 500 and the outlet portion 200 of the housing, providing the internal side wall of the chamber, and a portion of the external wall of the housing. Alternatively, for, example, the ring can be inserted within the internal diameter of the inlet portion, wherein the ring provides the internal side wall of the chamber, and the ring does not provide a portion of the external wall of the housing.

The housing can include a variety of configurations. In the illustrated embodiment shown in FIG. 2B, the inlet portion 100 includes an inlet portion wall 103 including an inner surface 104, including a slot 105, and a plurality of concentric ridges 106 and channels 107, wherein the ridges and channels are interrupted by the slot. In this illustrated embodiment, the slot varies in depth, having a greater depth at the end near the inlet port 101a, than at the other end of the slot. Similarly, in the illustrated embodiment shown in FIG. 2B, the outlet portion is identical to the inlet portion, and the outlet portion 200 includes an outlet portion wall 203 including an inner surface 204, including a slot 205, and a plurality of concentric ridges 206 and channels 207, wherein the ridges and channels are interrupted by the slot. In this illustrated embodiment, the slot varies in depth, having a greater depth at the end near the outlet port 201a, than at the other end of the slot.

In some embodiments of the device, the internal ring is arranged such that the received immunoglobulin binding media form a packed column-like matrix in the chamber within the housing. For example, the internal diameter and/or the height of the internal ring can be selected based upon one or more of the following regarding immunoglobulin binding particles to be used: the size, the volume, and/or the surface area, such that the immunoglobulin binding media passing through the inlet are retained by the mesh or screen or leukocyte depletion filter, and form a packed column-like matrix within the housing.

In another embodiment, a flexible housing, e.g., a flexible container, preferably a flexible container having at least two ports (such as a blood bag), can be used, and at least one filter element and/or immunoglobulin binding media can be disposed in the flexible container.

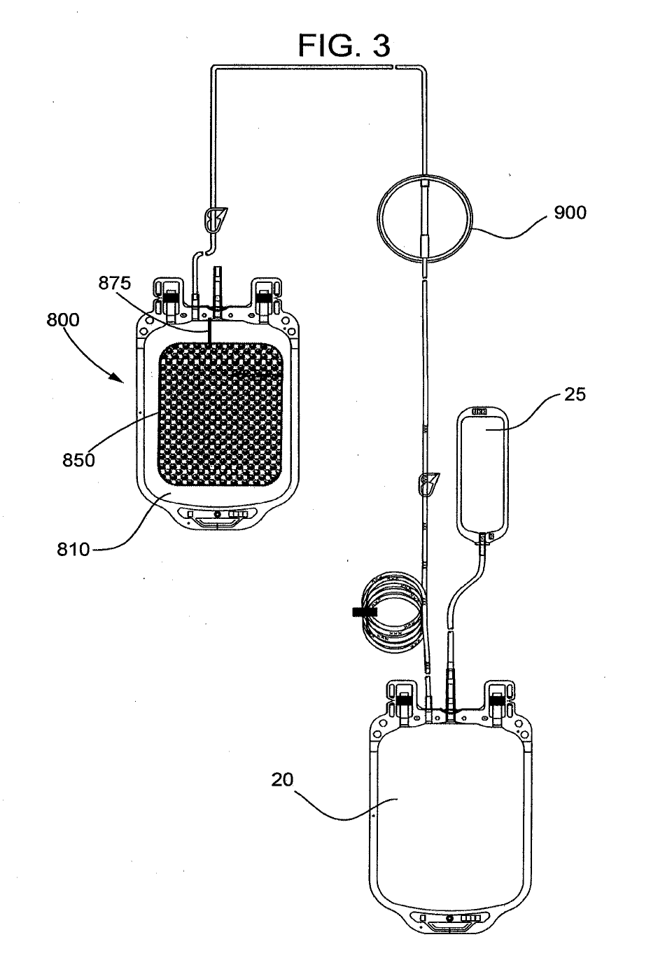

For example, immunoglobulin-specific binding media can be placed in a flexible or rigid container allowing biological fluid to be passed through a port into the container to allow the biological fluid to contact the binding media. The binding media can be loose in the container (e.g., loose in container 10 shown in FIG. 1), or, for example, retained in a pouch comprising a mesh or pores (e.g., as shown in FIG. 3, binding media device 800 comprises a flexible container 810 (such as a blood bag) containing a sealed pouch 850 comprising mesh walls containing beads of binding media therein (binding media not shown), the mesh preventing passage of the media from the pouch, FIG. 3 also illustrating a tether 875 connecting the pouch to the bag), or the container can include a porous element such as a screen or mesh that allows biological fluid to pass from the container and through a port without allowing the binding media to pass from the container.

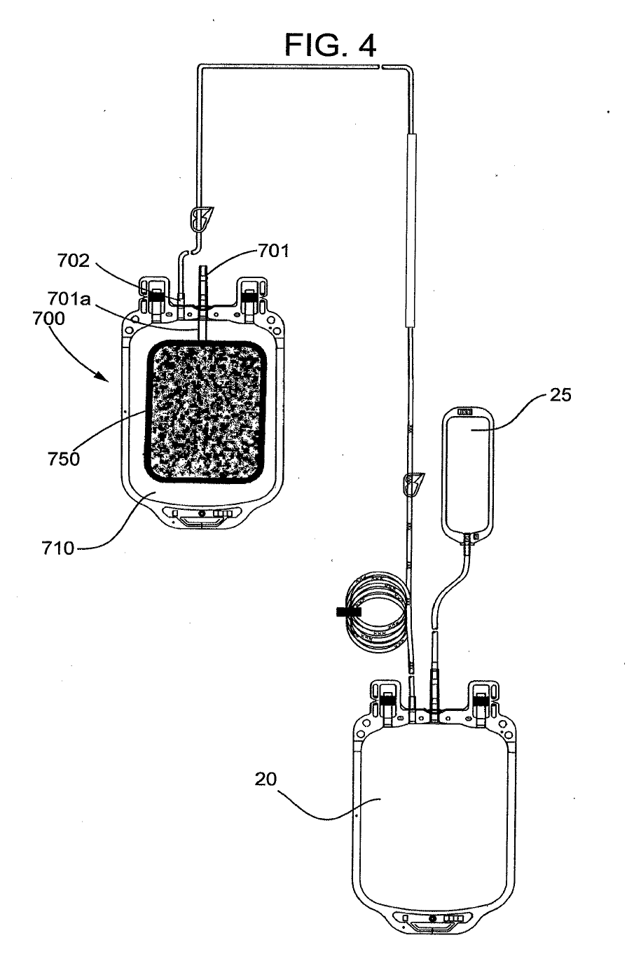

Alternatively, or additionally, at least one filter element can be disposed in a flexible container comprising at least two or more ports, at least a first port providing an inlet and at least a second port providing an outlet, wherein the first and second port define at least one fluid flow path between the inlet and the outlet, wherein the filter element is disposed across the fluid flow path, to provide a filter device. In one embodiment (e.g., as shown in FIG. 4, showing filter device 700, comprising a flexible container 710 (such as a blood bag) comprising a leukocyte depletion filter element 750 arranged to provide a chamber for immunoglobulin binding media therein (immunoglobulin binding media not shown), at least one filter element can be arranged in the general form of a pouch having a closed end and an open end, with binding media in the cavity of the pouch, and arranged in the flexible container between the inlet port and the outlet port such that biological fluid passes through the inlet, into the cavity of the pouch such that it contacts the binding media, and passes through the closed end of the filter element comprising a porous fibrous leukocyte depletion medium, and through the outlet. In the embodiment illustrated in FIG. 4, a conduit provides the inlet 701 and inlet port 701a, wherein the conduit passes through the open end of the filter element 750, and the filter element is sealed to the conduit such that biological fluid passes through the conduit into the cavity of the pouch. Fluid passing from the cavity and through the closed end of the filter element subsequently passes through the outlet 702, and in the illustrated embodiment, the outlet is not connected to the filter element.

Suitable flexible containers can be fabricated from, for example, polymeric materials such as films identical to or similar to those used in forming blood bags, such as plasticized polyvinyl chloride, plasticized ultra-high-molecular weight PVC resin, ethylene butyl acrylate copolymer (EBAC) resin, ethylene methyl acrylate copolymer (EMAC) resin, and ethylene vinyl acetate (EVA).

The housing (e.g., rigid or flexible) can be sealed as is known in the art, utilizing, for example, an adhesive, a solvent, laser welding, radio frequency sealing, ultrasonic sealing and/or heat sealing. Additionally, or alternatively, the housing can be sealed via injection molding.

Typically, the filter devices and immunoglobulin binding media according to the invention are included in a biological fluid processing system, e.g., a system including a plurality of conduits and containers, preferably flexible containers such as blood bags (e.g., collection bags and/or satellite bags). The biological fluid processing system can be suitable for processing colostrum. In one embodiment, a system according to the invention comprises a closed system, and the biological fluid can be processed while maintaining a closed system. A wide variety of suitable containers and conduits are known in the art. For example, blood collection and satellite bags, and conduits, can be made from plasticized polyvinyl chloride. Bags and/or conduits can also be made from, for example, ethylene butyl acrylate copolymer (EBAC) resin, ethylene methyl acrylate copolymer (EMAC) resin, plasticized ultra-high-molecular weight PVC resin, and ethylene vinyl acetate (EVA). The bags and/or conduits can also be formed from, for example, polyolefin, polyurethane, polyester, and polycarbonate.

Embodiments of the system can include additional components, such as one of more of any of the following: containers (preferably, flexible containers such as blood bags), connectors, sampling devices (e.g., flexible pouches and/or rigid containers), vents (e.g., gas inlets and/or gas outlets), and flow control devices (e.g., clamps and/or in-line devices such as transfer leg closures and/or valves), as is known in the art. For example, the embodiments of the system shown in FIGS. 1, 3, and 4 further comprise a sampling pouch, sufficient conduit length for providing a plurality of segments for sampling while maintaining a closed system, blood bags 10, 20, 710, and 810, and a plurality of flow control devices. Using the illustrated embodiments shown in FIGS. 1, 3, and 4, since, in preferred embodiments, the invention is carried out while maintaining a closed system, a container containing biological fluid or colostrum is, for example, sterile docked to the biological fluid processing system to provide fluid communication to container 10 (FIG. 1), container 810 (FIG. 3) or container 710 (FIG. 4). In the embodiment illustrated in FIG. 3, the system also includes a leukocyte depletion device 900 (e.g., a commercially available leukocyte depletion device) downstream of binding media device 800. Additionally, the embodiments of the system shown in FIGS. 1 and 3 further comprise a gas inlet 51, and a gas outlet 52. In those embodiments including a vent such as a gas inlet and/or a gas outlet (e.g., comprising a housing and at least one vent element disposed in the housing), a variety of materials are suitable for use as vent elements. Suitable elements, including hydrophilic microporous membranes and hydrophobic porous membranes, and vents, are disclosed in, for example, U.S. Pat. Nos. 5,126,054 and 5,451,321. Preferably, when used in accordance with a closed system, the gas inlet and gas outlet prevents the passage of bacteria therethrough, e.g., the gas inlet and gas outlet include a vent element having a bacterial blocking pore rating.

Embodiments of the invention are suitable for processing biological fluid, and colostrum. In the following discussion, while “biological fluid” is referred to, colostrum can also be processed the same way.

In accordance with the invention, biological fluid (e.g., whole blood, at least one blood component, or a blood product) is placed in contact with immunoglobulin-specific binding media, and passed through a porous fibrous leukocyte depletion medium. Preferably, the immunoglobulin-specific binding media comprise particles.

Typically, after placing the biological fluid in contact with particles in a container, the biological fluid is mixed with the particles, e.g., by inverting the container containing the biological fluid and the particles once or twice. In some embodiments, this may be preferable to agitating the container using reciprocating, orbital, rotator type (including 3-D rotator) agitators, or rotomixers, as well as shaker devices, as using such agitator or shaker devices, particularly over an extended period of time, can result in hemolysis and/or platelet activation.

In accordance with one embodiment of the method according to the invention, and using the exemplary system shown in FIG. 3 for reference, the biological fluid is placed in contact with immunoglobulin-specific binding media to obtain an immunoglobulin-depleted biological fluid, and the immunoglobulin-depleted biological fluid is subsequently passed through the porous fibrous leukocyte depletion filter to obtain an immunoglobulin- and leukocyte-depleted biological fluid.

In accordance with another embodiment of the method, and using the exemplary system shown in FIG. 4 for reference, the immunoglobulin-specific binding media and the porous fibrous leukocyte depletion medium are in the same container or housing, and the biological fluid is placed in contact with immunoglobulin-specific binding media and passed through the porous fibrous leukocyte depletion medium essentially simultaneously to obtain an immunoglobulin- and leukocyte-depleted biological fluid.

In yet another embodiment of the method, and using the exemplary system shown in FIG. 1 and the exemplary device shown in FIG. 2 for reference, the biological fluid is placed in contact with immunoglobulin-specific binding media in a first container, and the biological fluid and immunoglobulin-specific binding media are passed from the first container into a filter device comprising (a) a chamber for receiving immunoglobulin binding media and (b) a porous fibrous leukocyte depletion medium; such that the binding media is retained in the chamber, and the biological fluid passes through the porous fibrous leukocyte depletion medium, to obtain an immunoglobulin- and leukocyte-depleted biological fluid.

Immunoglobulin- and leukocyte-depleted biological fluid obtained according to the invention can be subsequently processed as is known in the art. For example, immunoglobulin- and leukocyte-depleted biological fluid can be stored, analyzed and/or administered to a subject.

The following examples further illustrate the invention but, of course, should not be construed as in any way limiting its scope.

### Example 1

This example demonstrates a device comprising a blood bag containing immunoglobulin-specific binding media and a porous fibrous leukocyte depletion medium therein removes immunoglobulins and leukocytes from whole blood.

Two types of leukocyte depletion filters are prepared, both types are constructed from multiple layers of fibrous leukocyte depletion media, wherein the media are prepared as generally described in U.S. Pat. No. 4,925,572. One type of filter has 6 layers of media, the other has 12 layers. About 15 grams of (4-MEP) HyperCel™ chromatography sorbent (Pall Corporation, NY) is placed in the center of the stack of layers, and the layers are folded over and heat-sealed at the edges, forming a “pouch” about 4 inches wide and about 6 inches high, wherein the open end of the pouch is sealed to the external wall of a conduit passing therethrough.

The pouch is placed in a 1000 mL blood bag to form a filter device as generally shown in FIG. 4.

A unit of whole blood is mixed with CP2D anticoagulant. Two 90 mL portions of blood are separated from the unit and the levels of leukocytes and immunoglobulins are determined.

The 90 mL portions are placed in the filter devices and placed on a rocker for 30 minutes. The treated blood is transferred to another blood bag and the residual levels of leukocytes and immunoglobulins are determined.

The 6 layer pouch provides for 99.1% leukocyte reduction, 94.8% IgG reduction, and 64.6% IgA reduction, in a recovered volume of 60.5 mL. The 12 layer tea bag provides for 99.8% leukocyte reduction, 94.6% IgG reduction, and 70.9% IgA reduction, in a recovered volume of 45.5 mL.

This example demonstrates a device containing both leukocyte depletion media and immunoglobulin-specific binding media can provide greater that 99% leukocyte removal and greater than 90% IgG removal.

### Example 2

This example demonstrates a system including a blood bag containing immunoglobulin-specific binding media and a downstream filter device comprising an immunoglobulin binding media chamber and a porous fibrous leukocyte depletion filter therein removes immunoglobulins and leukocytes from packed red blood cells (PRC), wherein the filter device also captures immunoglobulin-specific binding media passed from the blood bag.

A filter device as generally shown in FIG. 2 is obtained, comprising 3.5 inch diameter inlet and outlet housing portions, and a ring interposable between the housing portions, each made of polycarbonate. The filter includes a prefilter element and a porous fibrous leukocyte depletion element, wherein the elements are prepared as generally described in U.S. Pat. No. 4,925,572. A 35-40 micrometer polyethylene 3.5 inch diameter screen is placed on top of the prefilter element, and the filter and screen are sealed the housing, between the outlet portion and the ring.

About 30 grams (dry weight) of cellulose beads, (4-MEP) HyperCel™ chromatography sorbent (Pall Corporation, NY), is placed in a 1000 mL blood transfer bag, followed by about 10 mL of phosphate buffered saline (PBS).

A unit (about 350 mL) of 5 day old packed red blood cells in AS-3 additive solution is placed in the bag, and the bag is placed on a rotomixer set at 60 rpm for 15 minutes.

The bag is attached to the filter device, and the red blood cells are gravity filtered at a head height of 60 inches. The beads are retained in the immunoglobulin binding media chamber, and the filtered red blood cells passing through the device are collected, and analyzed using a flow cytometer.

The leukocyte content is reduced by about 5.17 log(5 log=99.999%), IgG is reduced by about 98%, IgA by about 81%, and IgM by about 42%, in a recovered volume of about 320 mL.

### Example 3

This example demonstrates a device comprising a blood bag containing immunoglobulin-specific binding media also removes cytokines from packed red blood cells. Thus, for example, if the biological fluid is stored before leukocyte depletion, allowing the level of cytokines to increase, processing biological fluid in accordance with an embodiment of the invention will remove immunoglobulins, leukocytes, and cytokines from the biological fluid.

This example also demonstrates a leukocyte depletion filter removes leukocytes but not a significant level of cytokines from packed red blood cells.

Two units of about 22-33 day old ABO compatible non-leukocyte-depleted red cell concentrate are pooled together, and divided into two approximately 300 mL aliquots.

One aliquot is sterile connected to a standard blood bag containing about 25-33 grams (dry weight) of cellulose beads, (4-MEP) HyperCel™ chromatography sorbent (Pall Corporation, NY) and about 10 mL of phosphate buffered saline (PBS). The red cells are mixed with the beads for about 45 minutes, and the red cells are subsequently passed from the bag and analyzed.

The other aliquot is passed, via gravity filtration at a head height of 60 inches through a BPF4™ High Efficiency leukocyte depletion filter (Pall Corporation, Port Washington, N.Y.), and the filtered fluid is collected and analyzed.

With respect to the aliquot placed in contact with the chromatography sorbent, interleukin 1-Beta (IL-1β) is reduced by about 45.7%, Interleukin-6 (IL-6) is reduced by about 26.9%, Interleukin-8 (IL-8) is reduced by about 57.1%, and Tissue Necrosis Factor-Alpha (TNF-α) is reduced by about 49.9%.

With respect to the aliquot passed through a leukocyte depletion filter, interleukin 1-Beta (IL-1β) is essentially not reduced, Interleukin-6 (IL-6) is essentially not reduced, Interleukin-8 (IL-8) is reduced by about 35.0%, and Tissue Necrosis Factor-Alpha (TNF-α) is reduced by about 7.5%.

### Example 4

This example demonstrates immunoglobulins and leukocytes can be removed from equine blood.

Volumes of equine whole blood are collected and mixed with CPD anticoagulant (500-600 mL of blood combined mixed with about 63-70 mL of CPD), and samples are taken to determine prefiltration levels of IgG and leukocytes.

Some volumes of the blood mixed with CPD are centrifuged at 5000 g for 5 minutes to prepare red cell concentrate (RCC), about 80% of the plasma is removed (typically, about 90-95% of the plasma is removed when preparing RCC, the extra volume of plasma is present to allow a sufficient amount of IgG to be present for subsequent analysis), and the red cells are resuspended in red cell additive solution (AS-3). Samples are taken to determine prefiltration levels of IgG and leukocytes in the RCC.

In one experiment, volumes of about 50 mL of anticoagulated whole blood and about 50 mL of plasma are placed in separate blood transfer bags, each bag containing about 22 grams (dry weight) of cellulose beads, (4-MEP) HyperCel™ chromatography sorbent (Pall Corporation, NY), and phosphate buffered saline (PBS), and after passing the blood or plasma into the bags, the bags are inverted once or twice. The bags of whole blood and plasma (and the beads) are maintained at room temperature for about 60 minutes. One set of whole blood/plasma bags is placed on a rotamixer, and one set is placed on a laboratory bench without mixing, for the 60 minutes.

In another experiment, about 100 mL of anticoagulated whole blood, and about 300 mL of RCC in additive solution are placed in separate blood transfer bags, each bag containing about 33 grams (dry weight) of cellulose beads, (4-MEP) HyperCel™ chromatography sorbent (Pall Corporation, NY), and phosphate buffered saline (PBS), and after passing the blood or RCC into the bags, the bags are inverted once or twice. The bag of whole blood and RCC (and the beads) are placed on a laboratory bench without further mixing, at room temperature.

Filter devices as described in Example 2 are obtained, and attached to the bags, and the anticoagulated whole blood and RCC are gravity filtered at a head height of about 45 inches. The beads are retained in the immunoglobulin binding media chambers, and the filtered blood and red blood cells passing through the device are collected, and analyzed (regarding residual leukocytes) using a flow cytometer. The levels of IgG are analyzed using an Enzyme-Linked Immunosorbent Assay (ELISA) and SDS-PAGE.

Most of the IgG is removed (91.6% and 92.8% IgG removal from whole blood and RCC, respectively, using 33 grams of beads; about 95% IgG removal using 22 grams of beads), and the results are similar with and without mixing using a rotamixer. The prefiltration concentrations of leukocytes in the whole blood and RCC are 5.23±0.38×10³ leukocytes/μL and 6.42±1.01×10³ leukocytes/μL, respectively. After filtration, the concentrations are reduced to 0.04±0.01 leukocytes/μL and 0.19±0.25 leukocytes/μL respectively, a removal of over 99.99%.

### Example 5

This example demonstrates a device comprising a blood bag containing immunoglobulin-specific binding media also removes prions from packed red blood cells.

A unit of 1-2 day old red cell concentrate (RCC) in red cell additive solution (AS-3) is obtained. About 50 mL of scrapie-infected hamster brain homongenate (SIHBH) in isotonic buffered saline (PBS) is added to about 200 mL RCC to provide an amyloid concentrate of about 2% (v/v). An aliquot of about 20 mL is taken to determine prefiltration levels of IgG, leukocytes, and amyloids in the RCC.

The RCC in additive solution is placed in a blood transfer bag containing about 30 grams (dry weight) of cellulose beads, (4-MEP) HyperCel™ chromatography sorbent (Pall Corporation, NY), the beads also having a branched polyprimary amine, N-(3-aminopropyl methacrylamide) (APMA, Polysciences, Warrington, Pa.) linked thereto, and phosphate buffered saline (PBS), and after passing the RCC into the bags, the bags are inverted once or twice. The bead- and RCC-containing bag is placed on a laboratory bench without further mixing at room temperature for about 45 minutes.

A filter devices as described in Example 2 is obtained, and attached to the bag, and the RCC is gravity filtered at a head height of about 45 inches. The beads are retained in the immunoglobulin binding media chamber, and the filtered red blood cells passing through the device are collected, and analyzed (regarding residual leukocytes) using a flow cytometer. The levels of IgG and amyloid proteins are analyzed using an Enzyme-Linked Immunosorbent Assay (ELISA) and SDS-PAGE.

The results show a significant removal of infectious amyloid proteins (over about 90% removal), as well as significant removal of IgA and IgG (about 79% and 98% removal, respectively), and of leukocytes (about 99.99% removal).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for removing immunoglobulins and leukocytes from a biological fluid, the method comprising:
(a) placing the biological fluid in contact with immunoglobulin-specific binding media; and,
(b) passing the biological fluid through a porous fibrous leukocyte depletion filter to obtain immunoglobulin- and leukocyte-depleted biological fluid.

2. A method for removing immunoglobulins and leukocytes from a biological fluid, the method comprising:
(a) placing the biological fluid in contact with immunoglobulin-specific binding media to obtain immunoglobulin-depleted biological fluid; and,
(b) passing the immunoglobulin-depleted biological fluid through a porous fibrous leukocyte depletion filter to obtain immunoglobulin- and leukocyte-depleted biological fluid.

3. The method of claim 1, wherein the immunoglobulin-specific binding media are beads.

4. The method of claim 3, comprising placing the biological fluid in contact with the beads in a flexible container.

5. The method of claim 4, comprising passing the biological fluid from the flexible container and through a filter device comprising a housing having an inlet and an outlet and defining a fluid flow path between the inlet and the outlet wherein the porous fibrous leukocyte depletion filter is disposed in the housing and across the fluid flow path.

6. The method of claim 5, wherein the porous fibrous leukocyte depletion filter has an upstream surface and a downstream surface, and the filter device further comprises a bead-receiving chamber upstream of the filter, and the method includes retaining the beads in the device as the biological fluid passes through the device.

7. The method of claim 1, wherein placing the biological fluid in contact with immunoglobulin-specific binding media also includes placing the biological fluid in contact with cytokine-specific binding media, wherein the immunoglobulin-specific binding media comprise cytokine-specific binding media.

8. The method of claim 1, carried out while maintaining a closed system.

9. The method of claim 1, comprising obtaining biological fluid from a human.

10. The method of claim 1, comprising obtaining biological fluid from an equine mammal.

11. A device for removing immunoglobulins and leukocytes from a biological fluid, the device comprising a biological fluid container containing immunoglobulin-specific binding media and a porous fibrous leukocyte depletion medium therein.

12. The device of claim 11, wherein the biological fluid container comprises flexible container having walls comprising a flexible film, and wherein the porous fibrous leukocyte depletion medium is arranged to provide a hollow structure having at least one closed end, the hollow structure containing the immunoglobulin-specific binding media therein.

13. A filter device comprising a housing including an inlet and an outlet and defining a fluid flow path between the inlet and the outlet, an internal ring, and a leukocyte depletion filter disposed in the housing across the fluid flow path, the leukocyte depletion filter having an upstream surface facing the inlet, and a downstream surface facing the outlet, the device further comprising a chamber for receiving immunoglobulin binding media passing through the inlet, the chamber having a side wall defined by the internal ring, wherein the chamber is arranged between the inlet and the upstream surface of the leukocyte depletion filter element.

14. The device of claim 13, further comprising a mesh element, providing a bottom wall of the chamber.

15. A system for removing immunoglobulins and leukocytes from a biological fluid, the system comprising:
(a) a biological fluid container, containing immunoglobulin-specific binding media therein;
(b) a leukocyte depletion device comprising a housing having an inlet and an outlet and defining a fluid flow path between the inlet and the outlet and having a porous fibrous leukocyte depletion filter disposed between the inlet and the outlet and across the fluid flow path;
wherein the leukocyte depletion device is downstream of, and in fluid communication with, the biological fluid container.

16. A method for reducing or preventing red cell hemolysis in horses comprising
(a) obtaining biological fluid or colostrum from a mare,
(b) depleting immunoglobulins and leukocytes from the biological fluid or the colostrum to obtain an immunoglobulin- and leukocyte-depleted biological fluid or an immunoglobulin- and leukocyte-depleted colostrum; and,
(c) administering the immunoglobulin- and leukocyte-depleted biological fluid or the immunoglobulin- and leukocyte-depleted colostrum to a foal.
